(19) United States
(12) Patent Application Publication
(10) Pub. No.: US 2004/0133189 A1
(43) Pub. Date: Jul. 8, 2004

(54) Surgical operation apparatus and control method thereof

(75) Inventor: Tomohisa Sakurai, Kanagawa (JP)

Correspondence Address: SCULLY SCOTT MURPHY & PRESSER, PC, 400 GARDEN CITY PLAZA, GARDEN CITY, NY, 11530

(73) Assignee: OLYMPUS CORPORATION, TOKYO (JP)

(21) Appl. No.: 10/743,224

(22) Filed: Dec. 22, 2003

(30) Foreign Application Priority Data
Dec. 27, 2002 (JP) 2002-381633

(51) Int. Cl. A61B 17/00

(52) U.S. Cl. 606/1; 606/34

(57) ABSTRACT

A surgical operation apparatus includes a surgical instrument; a drive device that generates an energy with which the surgical instrument works; and an energy transmission cable. The surgical operation apparatus also includes a first connector provided on one end of the cable; an energy release unit that releases the energy out of the first connector; a second connector that is detachable from the first connector; an energy receiving unit that receives the energy released from the energy release unit, and an operation functioning unit that functions based on the energy received.

### CROSS-REFERENCE TO RELATED APPLICATIONS

The present document incorporates by reference the entire contents of Japanese priority document, 2002-381633 filed in Japan on Dec. 27, 2002.

### BACKGROUND OF THE INVENTION

1) Field of the Invention

The present invention relates to a surgical operation apparatus, which is simple in manipulation and operation, and a control method of the surgical operation apparatus.

2) Description of the Related Art

Various kinds of operation apparatuses and systems for surgical operations have been developed. For example, ultrasonic cutting and coagulation apparatuses, electrosurgical knives, and bipolar treatment jigs, in which they vibrate with ultrasonic vibration to cut out or coagulate biomedical tissue, have been proposed.

In recent years, endoscopic surgical operations of reduced surgical cut and smaller invasion to a patient have been widespread. FIG. 10 is an illustration of a conventional surgical operation apparatus that a treatment jig for such endoscopic surgical operations is used. As illustrated in FIG. 10, with a surgical operation apparatus 1, a main body 3 provided with a connector port 2 detachably mounts thereto a hand piece 4 as a treatment jig via a connector 6 provided at an end of a transmission cable 5. An operator then operates an operation switch 7, which is connected to the main body 3, to manipulate the hand piece 4.

In such a surgical operation apparatus, when different kinds of hand pieces 4 are selected and used according to an object, an assistant first dismounts a connector that have already been connected to the connector port 2 of the main body 3, then selects another hand piece 4 according to an object, and connects a connector 6 on the cable 5 provided on the hand piece 4, to the connector port 2 of the main body 3 (see Japanese Patent Application Laid-open Publication No. 2000-271135).

In such an apparatus provided with a plurality of hand pieces according to objects, there is generated a need of exchanging hand pieces 4 conformed to operations several times. At this time, treatment jigs such as hand pieces with lengths of cable, are used in a state, in which they come and go at many time between a surgical bed and a sterilized instrument base, on which operative instruments required for operations are arranged and which is disposed close to the surgical bed.

Meanwhile, surgical robots in place of hands of an operator have been developed and applications of them to various surgical operations have been in practice. As illustrated in FIG. 11, an operator executes procedures such as removal of internal organs 16 by inserting an insert 13 mounted on a tip end of a manipulator 12 through an insertion hole 11 formed on a living body wall 10 and manipulating an endoscope 14 or a treatment jig 15 provided on a tip end of the insert 13 by means of remote manipulation (see Japanese Patent Application Laid-open Publication No. H7-136173)

### SUMMARY OF THE INVENTION

It is an object of the present invention to at least solve the problems in the conventional technology.

A surgical operation apparatus according to one aspect of the present invention includes a surgical instrument; a drive device that generates an energy with which the surgical instrument works; an energy transmission cable that has a first end and a second end, and that transmits the energy, the first end being connected to the drive device; a first connector provided on the second end; an energy release unit that releases the energy transmitted by the electric transmission cable out of the first connector; a second connector that is detachable from the first connector; an energy receiving unit that receives the energy released from the energy release unit, and an operation functioning unit that functions based on the energy received in the energy receiving unit.

A method according to one aspect of the present invention is of controlling a surgical operation apparatus. The surgical operation apparatus includes a first connector that releases energy generated by a drive device, a second connector that receives the energy, and a surgical instrument provided on the second connector. The method includes reading information of the surgical instrument from an identification information storage unit provided in the second connector by using a first information exchange unit provided in the first connector; and setting a drive state of the drive device based on the information.

The other objects, features and advantages of the present invention are specifically set forth in or will become apparent from the following detailed descriptions of the invention when read in conjunction with the accompanying drawings.

### BRIEF DESCRIPTION OF THE DRAWINGS

FIG. 1 is a schematic view of a surgical operation apparatus according to a first embodiment;

FIG. 2 is an enlarged view of an interconnection unit of the surgical operation apparatus;

FIG. 3 is a schematic view of the surgical operation apparatus;

FIG. 4 is a flowchart of a method of controlling the surgical operation apparatus;

FIG. 5 is a schematic view of a surgical operation apparatus according to a second embodiment;

FIG. 6 is a schematic view of a surgical operation apparatus according to a third embodiment;

FIG. 7 is a schematic view of a surgical instrument in a body cavity;

FIG. 8 is a schematic view of a surgical operation apparatus according to a fourth embodiment;

FIG. 9 is a schematic view of a surgical operation apparatus according to a fifth embodiment;

FIG. 10 is a schematic view of a conventional surgical operation apparatus; and

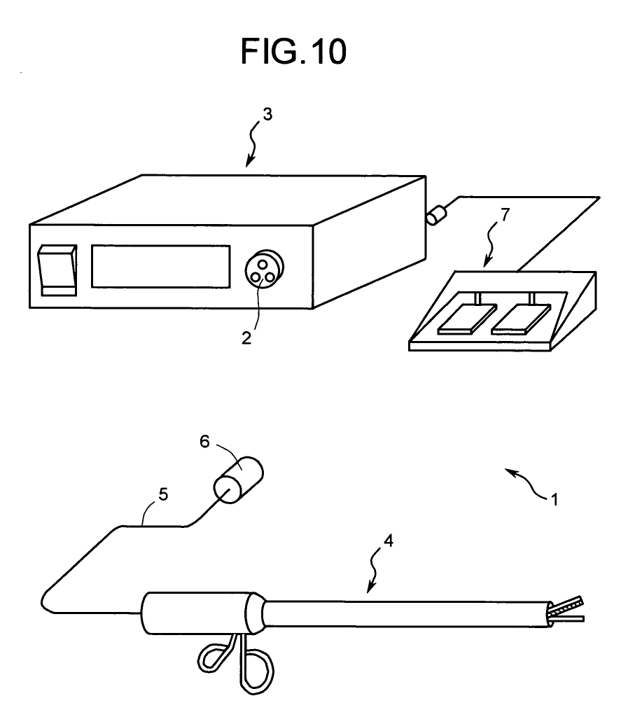

FIG. 11 is a schematic view of a conventional manipulator surgical instrument.

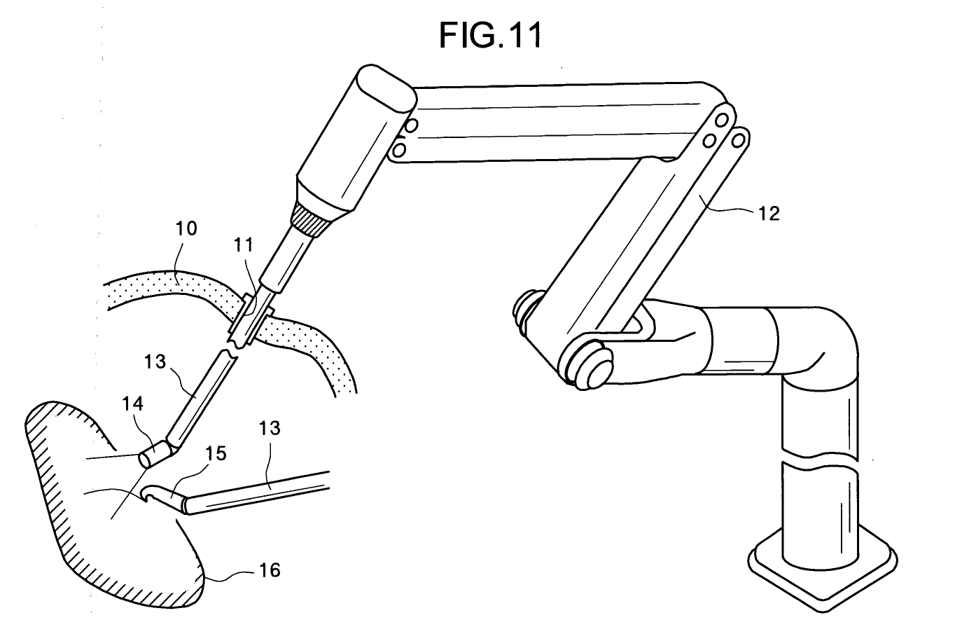

### DETAILED DESCRIPTION

Exemplary embodiments of a surgical operation apparatus relating to the present invention will be explained in detail below with reference to the accompanying drawings.

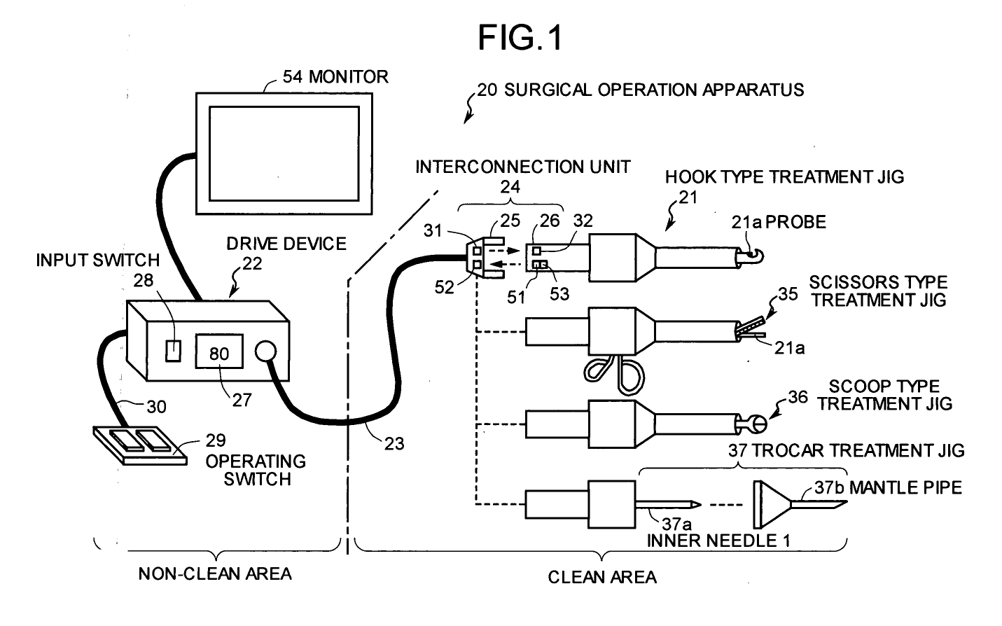

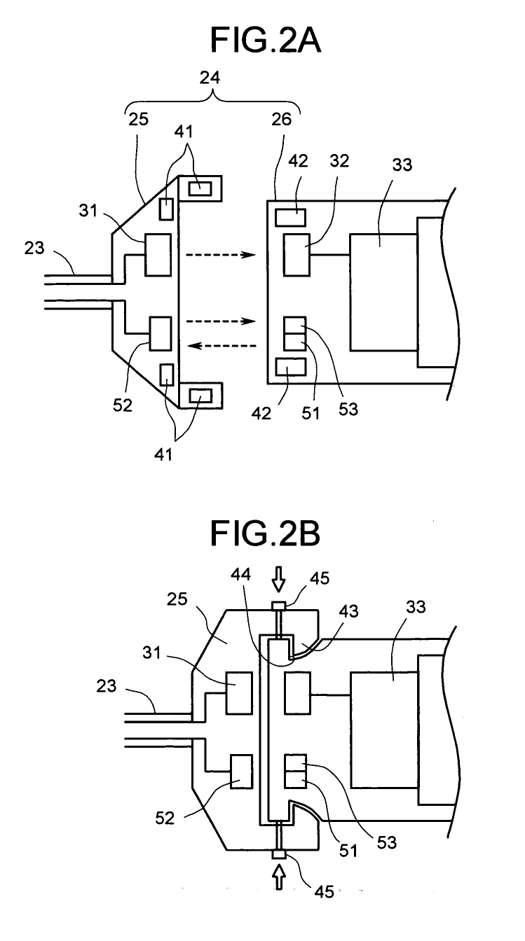

FIG. 1 is a schematic view of a surgical operation apparatus according to a first embodiment. FIG. 2 is an enlarged view of an interconnection unit. As illustrated in these figures, the surgical operation apparatus 20 includes surgical instruments used in operating a body (hereinafter, “examined body”). As typical of the surgical instruments, a hook type treatment jig 21 will be explained below. The hook type treatment jig 21 is provided with the probe 21 a, which is actuated by ultrasonic vibration used in endoscopic surgical operations. The surgical operation apparatus 20 also includes a drive device 22, an energy transmission cable (hereinafter, “cable”) 23, an energy transmission unit connector 25, a power supply coil 31, an energy receiving unit connector 26, a power receiving coil 32, and an operation functioning unit

The drive device 22 generates an energy required for functioning of the hook type treatment jig 21. The energy transmission cable 23 is connected at one end thereof to the drive device 22 to transmit the energy thereto. The energy transmission unit connector 25 is provided on the other end of the cable 23 and is referred to as a first connector. The power supply coil 31 is disposed within the energy transmission connector 25 and is an energy release unit that releases the energy transmitted by the cable outside of the connector 25 in the energy transmission unit. The energy receiving unit connector 26 is provided on the hook type treatment jig 21 and is referred to as a second connector that is detachable from the energy transmission unit connector 25. The power receiving coil 32 is disposed within the energy receiving unit connector 26 to receive the energy released from the power supply coil 31. The operation functioning unit is provided on the hook type treatment jig 21 that has a probe 21 a that functions based on the energy received in the power receiving coil 32.

In other words, by an interconnection unit 24 that includes the energy transmission unit connector 25, which has no electric contact and transmits and receives energy, and the energy receiving unit connector 26, the hook type treatment jig 21 and the cable 23 are coupled to each other, and energy is supplied to the hook type treatment jig 21 in a contact-free manner.

The surgical instruments are, for example, medical peripheral equipments, such as lighting equipments and endoscopes, which function for surgical operation treatments, in addition to treatment jigs, such as scissors, scalpels, hooks, and drills, which treat a living body as an object in surgical operation.

In addition, in ultrasonic cutting and coagulation, ultrasonic vibration generated by an ultrasonic vibrator is transmitted to a biomedical tissue via the probe to vibrate, whereby the softened tissue is fused and adequate coagulation is achieved by frictional heat due to the ultrasonic vibration. That is, rapid cutting-out is made possible by heat due to friction and mechanical abrasion.

In the first embodiment, for example, a scissors type treatment jig 35, a scoop type treatment jig 36, a trocar type treatment jig 37 composed of an inner needle 37a and a mantle pipe 37b, a suction treatment jig (not illustrated) can be exemplified as illustrated in FIG. 1, besides the hook type treatment jig 21, a surgical instrument, but these are not limitative.

As illustrated in FIG. 2, the interconnection unit 24 includes the energy transmission unit connector 25 as the first connector provided on an cable end, through which energy from the drive device 22 is supplied, and the energy receiving unit connector 26 as the second connector provided on a side of the surgical instrument for reception of the energy, these elements being detachably connected to each other by a contact-free arrangement.

As illustrated in FIG. 2, in the energy transmission unit connector 25, the power supply coil 31 is disposed as an energy release unit. In the energy receiving unit connector 26, the power receiving coil 32 is disposed as an energy receiving unit, and an ultrasonic vibrator 33 supplied with energy from the power receiving coil 32 to generate ultrasonic vibration is disposed. Electric energy from the power receiving coil 32 is converted into ultrasonic vibration in the ultrasonic vibrator 33 to be transmitted to the probe 21 a to vibrate it, frictional heat generated by vibration at the tip end of the probe coagulates biomedical tissue, and mechanical vibration of the probe cuts out the biomedical tissue.

The contact-free interconnection unit 24 is used to achieve fitting of the energy transmission unit connector 25 and the energy receiving unit connector 26 whereby the power supply coil 31 as a primary coil and the power receiving coil 32 as a secondary coil are fixed with axes thereof being in agreement with each other. The drive device 22 feds electricity to the power supply coil 31 to generate magnetic flux in the power supply coil 31, and electric current is generated in the power receiving coil 32 due to electromagnetic induction thereof. Thereby, energy is supplied to the ultrasonic vibrator 33 to generate ultrasonic vibration to permit the probe 21 a of the hook type treatment jig 21 to perform procedures such as cutting and coagulation.

According to the first embodiment, since electromagnetic induction makes it possible to transmit energy to the hook type treatment jig 21 in a contact-free manner, the contact-free interconnection unit 24 for mounting and dismounting of the hook type treatment jig 21 can be enhanced in insulation performance and the mounting and dismounting is made simple to improve operability. Since the hook type treatment jig can be exchanged at an end of the cable 23 extended from the drive device 22, an operator can exchange a desired surgical instrument based on one's own intention in a clean area and such exchange can be simply and rapidly performed. Accordingly, stresses of an operator in endoscopic surgical operation are dissolved.

Since the interconnection unit 24 can be structured to be insulative and flat, it is not complicated in structure and so can be readily and surely effectuated even in, for example, a severe sterilization-equipment such as autoclaves.

Since there is no need for a complicate waterproof construction like a contact construction and there is no need for a construction making use of expensive contacts made of a material, for example, gold, capable of withstanding the autoclave sterilization, manufacture can be made simple and inexpensive.

In the first embodiment, as illustrated in FIG. 2A, a first magnetism generation unit 41 is provided in the energy transmission unit connector 25, and a second magnetism generation unit 42 is provided in the energy receiving unit connector 26 to generate magnetism matching with that generated in the first magnetism generation unit 41, whereby the connectors are firmly joined with each other to eliminate easy disconnection during operation. These magnets may include electromagnets.

The magnetic coupling unit may further include a magnetism generation control unit capable of controlling magnetism generated in the first magnetism generation unit 41 and magnetism generated in the second magnetism generation unit 42, and thus magnetic forces as generated may be controlled.

As illustrated in FIG. 2B, pawls 43 provided in the energy transmission unit connector 25 of the interconnection unit 24 and pawls 43 provided in the energy receiving unit connector 26 may constitute an engagement unit 44, and an engagement release unit 45 may be provided to effect connection and disconnection to assure engagement and release.

In the first embodiment, a identification information storage unit (for example, IC chip) 51 is disposed in the energy receiving unit connector 26 to be able to store identification information indicative of a surgical instrument, and a first information exchange unit 52 is disposed in the energy transmission unit connector 25 to have information stored in the identification information storage unit 51 and to issue outside of the energy transmission unit connector 25 a signal for reading of information stored in the identification information storage unit 51. Further, arranged in the energy receiving unit connector 26 is a second information exchange unit 53 to receive a signal issued from the first information exchange unit 52 to have information stored in the identification information storage unit 51 and to read information stored in the identification information storage unit 51, so that a type of and individual information of a surgical instrument are instantaneously recognized by radio.

Meanwhile, in the drive device 22 for energy supply as illustrated in FIG. 1, a display unit 27 for displaying an output of the drive device and an input switch 28 for an operating input such as output of the drive device 22, are provided. Also provided on and connected to the drive device 22 via the cable 30 is an operating switch 29, by means of which an operator optionally operates an output state of the hook type treatment jig 21 as a surgical instrument.

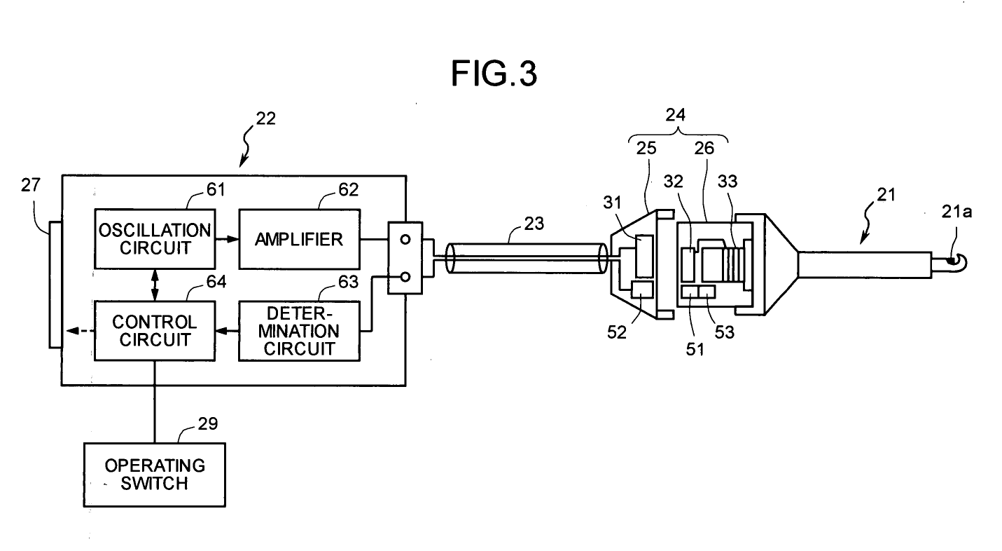

As illustrated in an electric circuit diagram of FIG. 3, the drive device 22 in the first embodiment includes an oscillation circuit 61 for generating a drive signal for driving of the ultrasonic vibrator 33, an amplifier 62 for amplifying a drive signal from the oscillation circuit 61, a determination circuit 63 for reading internal information in the identification information storage unit 51 from the information exchange unit 52 to execute determination, or for writing new information, and a control circuit 64 as a control unit that controls the oscillation circuit 61 based on information from the determination circuit 63 and controls other circuits in the device. Thereby, a drive parameter of the oscillation circuit 61 conformed to the characteristics of a treatment jig is automatically set based on information from the information exchange unit to enable appropriately supplying energy for medical treatment to the ultrasonic vibrator 33.

The drive device 22 does not adopt a construction including any isolation transformer inside like the conventional art, but the power supply coil 31 is arranged in the energy transmission unit connector 25 to provide isolation between the drive device 22 and the hook type treatment jig 21.

In carrying out the ultrasonic operation with the use of such device, when the energy transmission unit connector 25 at the end of the cable 23 extended from the drive device 22 is coupled to the energy receiving unit connector 26, the information exchange unit 52 reads information peculiar to the hook type treatment jig 21 from the identification information storage unit 51 by radio. The control circuit 64 recognizes a type of a treatment jig as, for example, a hook type treatment jig based on information read by the information exchange unit 52 to set a drive condition in the oscillation circuit 61 in a state suited to driving of the probe 21 a. In other words, many types of treatment jigs are present in the surgical instrument, and output capable of exhibiting an individual performance is determined every jig. Accordingly, it becomes important to instantaneously determine such individual information in the drive device 22.

As illustrated in FIG. 3, when an operator operates the operating switch 29 to indicate start of driving, the oscillation circuit 61 is driven and a drive signal from the oscillation circuit 61 is amplified by the amplifier 62 to be fed via the cable 23 to the power supply coil 31 in the energy transmission unit connector 25. Such energy is received by the power receiving coil 32 in the energy receiving unit connector 26 in a contact-free manner to be transmitted to the ultrasonic vibrator 33. Thereby, the ultrasonic vibrator 33 generates ultrasonic vibration, which is transmitted to the probe of the hook type treatment jig 21 to enable performing cutting and coagulation on a living body at the tip end thereof.

The control circuit counts that duration, during which ultrasonic vibration is generated by the operating switch 29, or the number of outputting, and such information is transmitted to the information exchange unit 52 to be fed to the identification information storage unit 51 by radio to be written in a memory provided therein.

It suffices that a control method for the surgical operation apparatus constructed in the above-mentioned manner include using the first information exchange unit 52 to read information stored in the identification information storage unit 51, and thereafter setting a drive state of the drive device 22 based on the read information to thereby effect control efficiently.

The control method for the surgical operation apparatus may include stopping reading of information by the first information exchange unit 52 after a drive state of the drive device 22 is set, and causing the drive device 22 to generate the energy based on the set drive state after reading of information by the first information exchange unit 52 is stopped.

The control method for the surgical operation apparatus may include detecting drive information of the drive device 22, using the first information exchange unit 52 to release the drive information detected in the drive information detecting step, receiving the released drive information in the second information exchange unit 53, and storing the received drive information in the identification information storage unit 51.

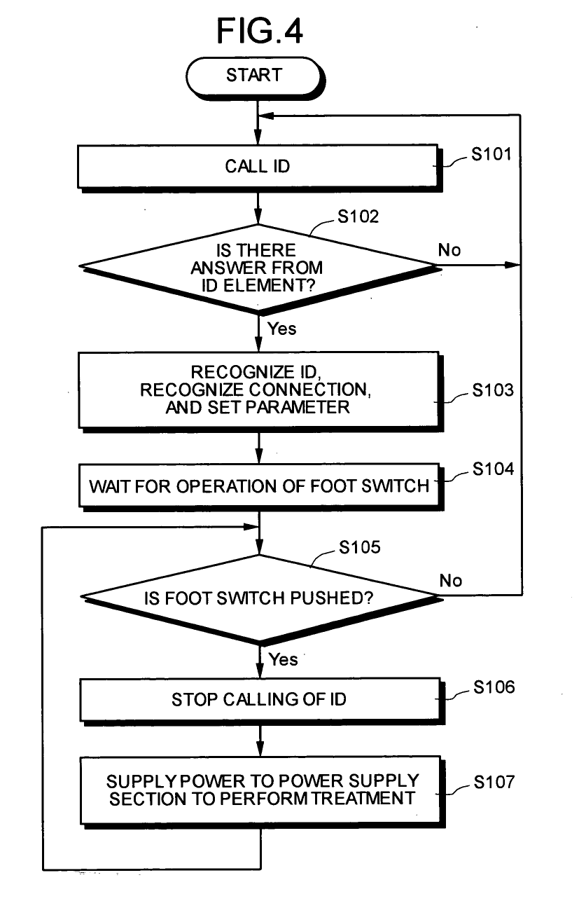

An example of a concrete flowchart of control in the first embodiment will be described next with reference to FIGS. 3 and 4.

First, when a power source for the drive device 22 is made ON, the determination circuit 63 calls an ID to the identification information storage unit (ID element) 51 via the information exchange unit 52 by radio (step S101).

It is then determined whether an answer comes from the identification information storage unit 51 (step S102). When there is no answer, the ID is again called.

When an answer is present (Yes at step S102), the ID is recognized, it is then recognized that the interconnection unit 24 has been connected, and a parameter peculiar to the hook type treatment jig 21 is set in the drive device 22 (step S103).

Next, operation of the operating switch 29 by an operator is waited (step S104).

Presence and absence of operation of the operating switch 29 by an operator is then confirmed (step S105). When operation by an operator is present (Yes at step S105), calling of ID is stopped (S1 06).

When operation by an operator is absent (No at step S105), the procedure is returned to START.

When operation of the operating switch 29 is detected and calling of ID is stopped, energy is supplied to the energy transmission unit connector 25 from the drive device 22 to be supplied to the energy receiving unit connector 26 in a contact-free manner, and the treatment is performed by the hook type treatment jig 21 (step S107).

The operation is repeated whenever a plurality of surgical instruments is exchanged, and output is appropriately set according to a type of the exchanged surgical instrument. In addition, such output can be suitably displayed on the display unit 27 of the drive device 22 or a monitor 54 connected to the drive device (see FIG. 1). In particular, since the monitor 54 displays image information fed from the endoscope and a type of a presently used surgical instrument together therewith, the surgical instrument in use can be confirmed, so that an operator can instantaneously make a determination.

A surgical instrument is in some cases changed in setting of output according to an operator's preference, habit, or the like to be used, in which case output may be set based on information of a particular operator by storing such information in the identification information storage unit 51 and identifying an operator in the drive device 22.

According to the first embodiment, an operator can optionally exchange plural types of surgical instruments in a clean area and a sure and inexpensive connection construction can be provided even in a severe working environment, such as washing of surgical instruments, sterilization process, or the like, essential for surgical operations.

After connection of a surgical instrument, its individual information can be presented to the information exchange unit 52 from the identification information storage unit 51 by radio and the information can be exactly transmitted to an operator by means of the displays 27, 54, or the like, occurrence of taking a wrong surgical instrument is beforehand prevented and output conformed to an associated surgical instrument can be supplied from the drive device 22.

While the first embodiment has been described with respect to the surgical instrument used in endoscopic surgical operation, the invention is not limited thereto but can be used in surgical instruments, which receive energy to treat a living body in general surgical operations.

Energy transmitted within the interconnection unit 24 may be not only electric energy but also other different energy, for example, light energy. For example, the surgical operation apparatus 20 illustrated in FIG. 1 may be provided therein with a first energy conversion unit may be disposed inside the energy transmission unit connector 25 as the first connector and for converting a first electric energy transmitted by way of the electric transmission cable 23 into a second different energy to release the energy outside the energy transmission unit connector 25, and a second energy conversion unit disposed inside the energy receiving unit connector 26 as the second connector provided on the surgical instrument and for receiving the second energy released by the first energy conversion unit to convert it into electric energy.

In other words, the hook type treatment jig 21 as a surgical instrument may be actuated by providing a light irradiation unit in place of the power supply coil 31, providing a light receiving unit in place of the power receiving coil 32, and providing an energy conversion unit that converts the received light energy into electric energy.

As the surgical instrument, a laser scalpel with a laser light emitting diode, a microwave scalpel making use of microwave, a thermal scalpel with a heater element, an electric drill, of which a cutting blade is rotated by a motor, or the like may be used.

A second embodiment of the invention will be described next. In the second embodiment, ultrasonic vibration as in the first embodiment is not used, and an explanation will be given when the surgical instrument is a bipolar treatment jig.

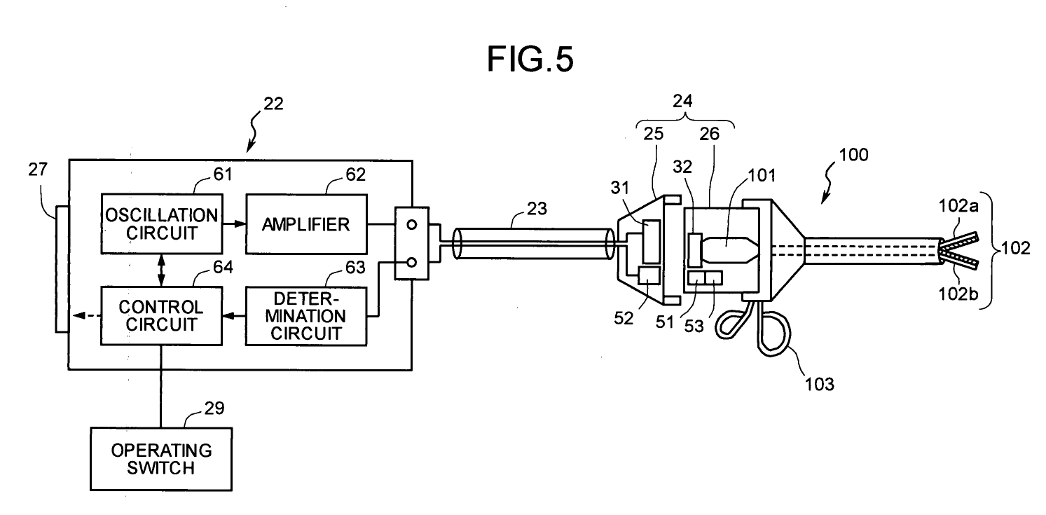

FIG. 5 is a schematic view of a surgical operation apparatus according to the second embodiment.

It should be noted that constituents like the constituents in the first embodiment are denoted by the like reference numerals, and an explanation therefor is omitted. The respective functions illustrated in the first embodiment may be added to the second embodiment.

As illustrated in FIG. 5, a bipolar treatment jig 100 as a kind of surgical instrument includes a medical current generation unit 101 for generating a high-frequency medical current according to energy received by the power receiving coil 32 as an energy receiving unit, and a medical electrode 102, to which current generated in the medical current generation unit 101 is transmitted and which is capable of performing a high-frequency treatment on an examined body based on the current, and the energy receiving unit connector 26 is provided with the identification information storage unit 51 capable of storing the identification information. Provided on a tip-end treatment unit on the bipolar treatment jig are a set of medical electrodes 102a, 102b insulated from each other. The respective electrodes 102a, 102b can open and close in accordance with the opening and closing operation of the handle 103.

When such an apparatus is used to perform surgery, a distance between the identification information storage unit 51 and the information exchange unit 52 decreases when connection is effected on an interconnection unit 24 of the bipolar treatment jig 100, and the identification information is read by radio communication.

The control circuit 64 sets a drive condition in the oscillation circuit 61 recognizing a type of surgical instrument as a bipolar treatment jig.

An operator operates the operating switch 29 to cause the amplifier 62 to amplify a drive signal from the oscillation circuit 61 to feed it to the power supply coil 31 in the energy transmission unit 25 via the cable 23. The energy is received by the power receiving coil 32 of the energy receiving unit connector 26 in a contact-free manner, the current is transmitted to the electrodes 102a, 102b in the tip-end treatment unit, and the bipolar current performs such treatment as cutting and coagulation on a living body.

Operating time or the number of output of the bipolar current indicated by the operating switch 29 is counted by the control circuit, and information thereof is transmitted to the first information exchange unit 52 and written in a memory in the identification information storage unit 51 by radio.

It is possible to exchange the bipolar treatment jig and the ultrasonic vibration treatment jig with each other as necessary, thus enabling performing an appropriate surgical operation treatment.

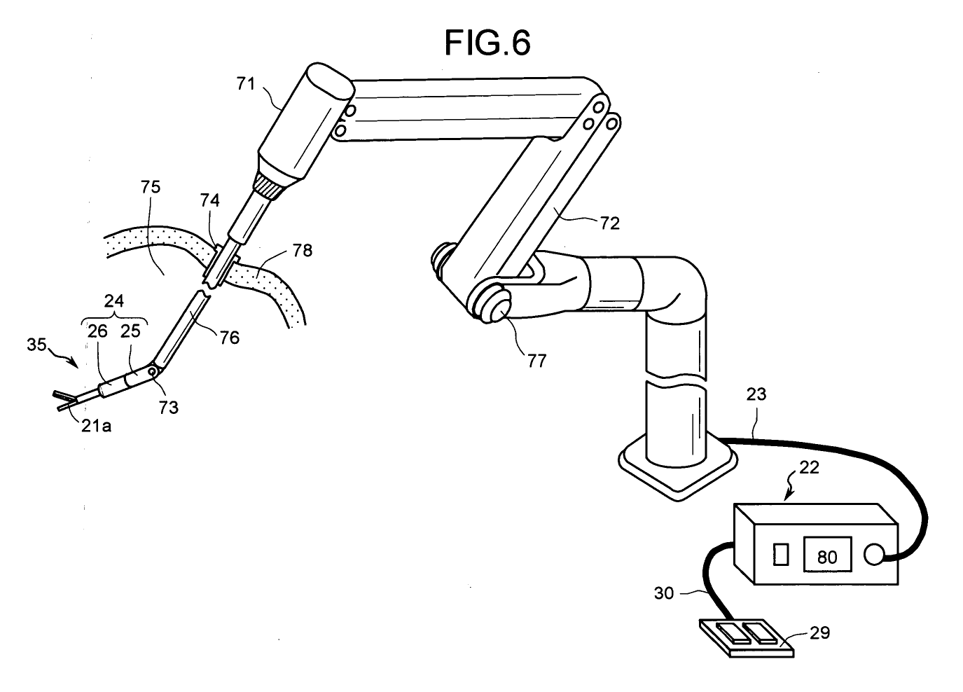

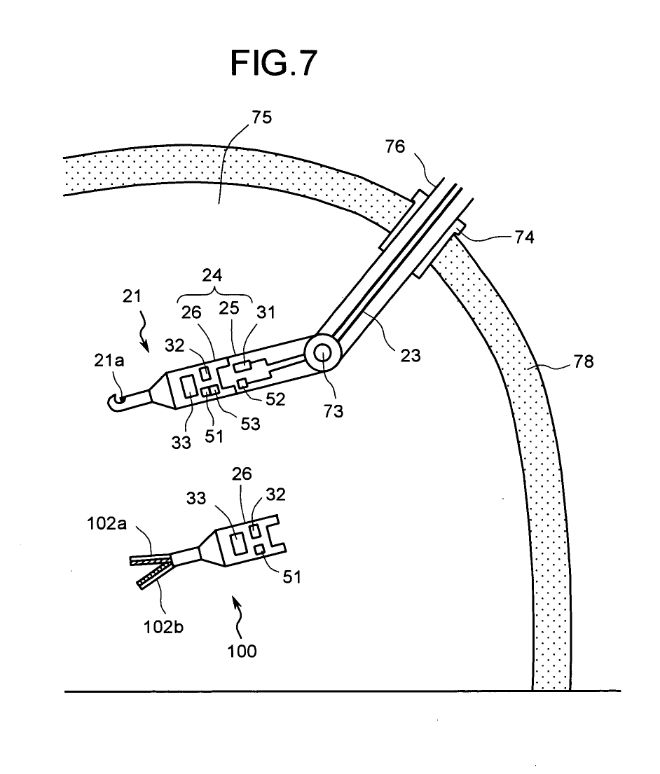

FIG. 6 is a schematic view of a surgical operation apparatus applied to a surgical manipulator according to a third embodiment. FIG. 7 is a schematic view of a tip-end medical unit of the apparatus.

It should be noted that constituents like the constituents in the first embodiment are denoted by the like reference numerals, and an explanation therefor is omitted. The respective functions illustrated in the first embodiment may be added to the third embodiment.

A manipulator for surgical operations, according to the third embodiment, having a remote operation unit that performs a remote operation, and controls a surgical instrument, by which a surgical operation is performed, based on a command from the remote operation unit to perform a surgical operation on an examined body, includes a drive device 22 for generating an energy required for functioning of a surgical instrument, for example, a scissors type treatment jig 35, an energy transmission cable (not illustrated) connected at one end thereof to the drive device 22 to transmit the energy thereto, a manipulator body 72 having an arm 71 movable to any position in a space of surgical operations, in which the energy transmission cable is arranged and the surgical instrument is used to perform a surgical operation, an energy transmission unit connector 25 provided on the other end of the energy transmission cable and being a first connector arranged on the arm, a power supply coil 31 disposed within the energy transmission unit connector 25 and being an energy release unit that releases the energy transmitted by the energy transmission cable outside of the first connector, an energy receiving unit connector 26 provided on the surgical instrument and being a second connector detachable from the energy transmission unit connector 25, a power receiving coil 32 disposed within the energy receiving unit connector 26 to receive the energy released from the power supply coil 31, and a probe 21 a provided on the surgical instrument and constituting an operation functioning unit that functions based on the energy received in the power receiving coil 32.

In other words, the manipulator for surgical operations, according to the third embodiment includes the manipulator body 72 having the arm 71 movable three-dimensionally, the drive device 22 for supplying energy to the scissors type treatment jig 35 as a surgical instrument, and an interconnection unit 24 composed of the energy transmission unit connector 25 and the energy receiving unit connector 26, which are provided on a tip end of the arm 71 with a linkage 73 therebetween. Like the first embodiment, since the interconnection unit 24 is constructed such that an end of the cable 23 connected to the drive device 22 is detachably coupled to the scissors type treatment jig 35 as a surgical instrument and energy is transmitted and received without the provision of electric contacts, it is possible to perform operations while simply and rapidly exchanging an appropriate surgical instrument being provided on the tip end of the manipulator according to contents of treatment.

In the third embodiment, a straight insertion arm 76 being inserted into a body cavity 75 through a trocar 74 provided in a living body wall 78 is provided, and the manipulator body 72 is fixed by a fixation unit (not illustrated).

Provided on the manipulator body 72 is a linkage mechanism 77 for positioning a tip end portion of the insertion arm 76 in the body cavity 75 and controlling a position thereof. The linkage mechanism 77 is controlled in position by a control unit (not illustrated). In addition, the third embodiment achieves operations in a master-slave system with a master arm (not illustrated), which an operator operates by hand, but is not limited thereto.

In the third embodiment, a linkage mechanism 73 is provided also on a tip end of the insertion unit in the body cavity 75, and the scissors type treatment jig 35 as a working unit can freely change a position of the tip end in the body cavity 75.

In the third embodiment, the scissors type treatment jig as a so-called end effector can be detachably provided on the interconnection unit 24 detachably arranged on the tip end of the insertion arm 76. In addition, various surgical instruments illustrated in FIG. 1 and described in the first embodiment can be used as an end effector.

FIG. 7 illustrates a state, in which the hook type treatment jig 21 capable of mounting and dismounting is connected to the tip end of the insertion arm 76 and another bipolar treatment jig 100 has already been inserted into the body cavity 75.

As illustrated in FIG. 7, the hook type treatment jig 21 is provided with a identification information storage unit 51, a second information exchange unit 53, a power receiving coil 32, which receives electric energy, and an ultrasonic vibrator 33. Also housed in the tip end of the insertion arm 76 on a side of the manipulator body are a power supply coil 31 and a first information exchange unit 52 for reading and writing information written or stored in the identification information storage unit 51.

As illustrated in FIG. 7, another surgical instrument is beforehand inserted as an end effector on the tip end into the body cavity 75 so that the bipolar treatment jig 100 as illustrated in FIG. 5 and the hook type treatment jig 21 as illustrated in FIG. 1 can be suitably exchanged with each other in operations. For example, lighting equipments and endoscopes in addition to surgical instruments may be beforehand inserted into the body cavity 75 and made detachable so that they can be used properly according to a type of a surgical instrument.

Upon connection of respective treatment jigs, the first information exchange unit 52 reads the identification information storage unit 51 provided on the respective treatment jigs, and consequently energy optimum for the respective treatment jigs as connected is fed to the power supply coil 31 from a control body (not illustrated). When the power receiving coil 32 provided on a side of an associated treatment jig receives power, respective treatments are performed.

According to the third embodiment, small-sized and waterproof connection is enabled to perform power transmission required for energy treatment and information exchange in a contact-free manner, and severe autoclave sterilisation or the like can be coped with.

Further, since the connection structure is made contact-free and simple, it is possible to achieve a surgical operation apparatus affording exchange of treatment jigs at a tip end thereof in a body cavity.

When such treatment units at a tip end of the apparatus are constituted by energy treatment jigs such as electrosurgical knife treatment units, ultrasonic cutting and coagulation apparatuses, according to the invention, it is possible to readily and surely recognize what treatment jig is connected to a tip end of a manipulator.

Particularly, in the case of exchange of treatment jigs in a body cavity, it is possible to achieve recognition of a treatment jig and transmission of power while ensuring a waterproof structure.

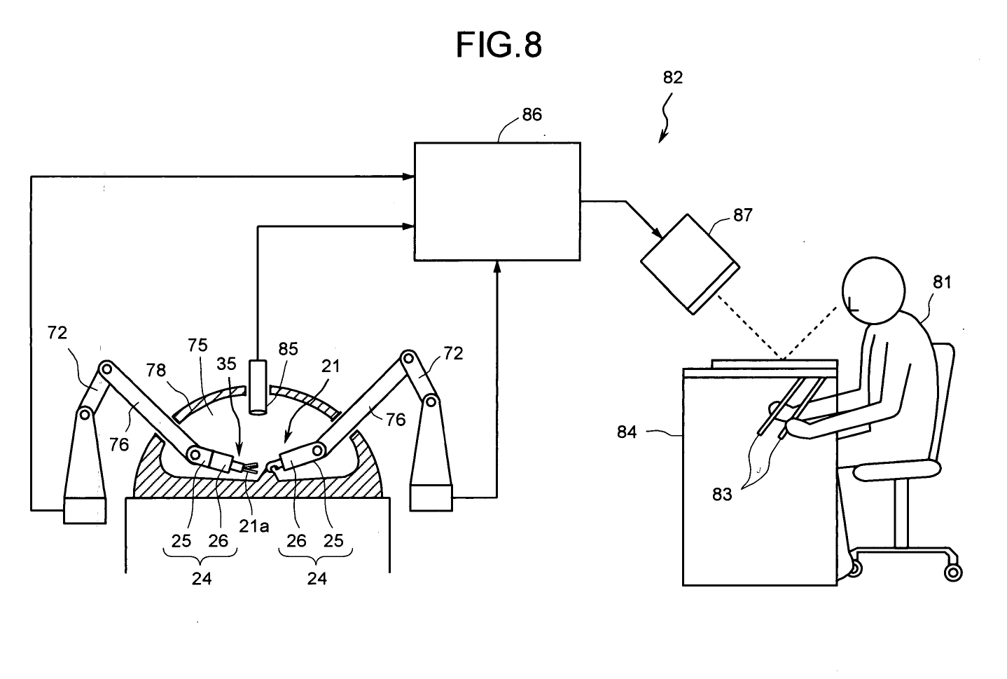

FIG. 8 is a schematic view of a surgical operation apparatus according to a fourth embodiment. The fourth embodiment adopts a manipulator apparatus provided with the same multiarticular arm as that illustrated in FIG. 7. It should be noted that constituents like the constituents in the first embodiment are denoted by the like reference numerals, and an explanation therefor is omitted. The respective functions described in the first embodiment may be added to the fourth embodiment.

As illustrated in FIG. 8, according to the fourth embodiment, provided in an operator station 84 arranged at a remote site of a remote manipulation system 82, by means of which an operator 81 operates a patient placed in a working space at a remote working site, is a hand controller 83 for performing remote control of a manipulator 72, which manipulates a surgical instrument for treatment on an object in the working space. An image acquisition device 85 is provided in a body cavity 75 to acquire internal images thereof, the image information is subjected to information processing in an information processing unit 86, and visible real-time images are reproduced in an image output device 87 arranged at the remote site. With the use of the information processing unit 86, which collects positional information of the hook type treatment jig 21 and the scissors type treatment jig 35 to subject the information to information processing, the operator 81 can manipulate the hand controller 83 to freely manipulate a surgical instrument as if the operator sees the body cavity as a working space with a real ambience. The interconnection unit 24 illustrated in the first embodiment provides the surgical instrument detachably on the arm tip end in a contact-free manner.

At this time, a surgical instrument provided with the energy receiving connector 26 may be beforehand placed in a body cavity, a surgical instrument provided with the energy receiving connector 26 of the interconnection unit 24 is separately supplied into a body cavity by means of a separate manipulator, and a manipulator (not illustrated) is used to optionally exchange a desired surgical instrument for application of a desired surgical operation. An identifying unit may be also housed in a prehension forceps, which holds a living body.

Thereby, it is possible to provide the remote manipulation system 82, which makes a surgical instrument detachable in a remote surgical operation making use of images, and in which surgical instruments are beforehand inserted in a body cavity to eliminate depositing and withdrawing of surgical instruments and operations can be rapidly performed.

By inserting another surgical instrument into a body cavity 75 with the use of a separate manipulator and thereafter exchanging the surgical instrument arbitrarily, an operator can perform a treatment rapidly while recognizing a desired surgical instrument based on identification information of a surgical instrument as exchanged, thus enabling an improvement in surgical operations.

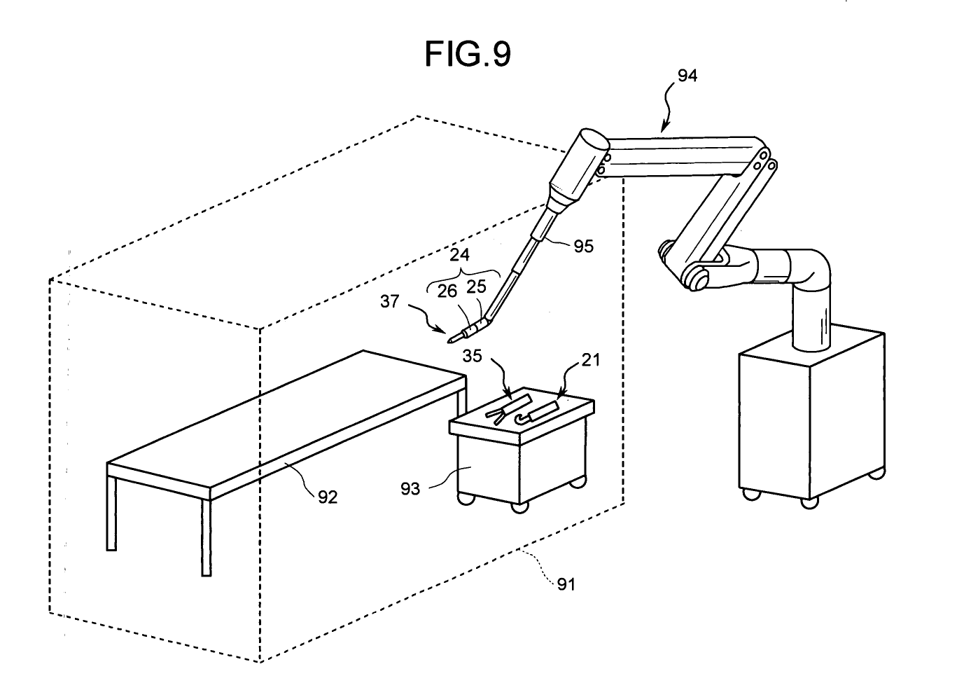

FIG. 9 is a schematic view of a further surgical operation system according to a fifth embodiment.

Constituents like the constituents in the first embodiment are denoted by the like reference numerals, and an explanation therefor is omitted. The respective functions illustrated in the first embodiment may be added to the fifth embodiment.

As illustrated in FIG. 9, a surgical operation apparatus according to the fifth embodiment is used to perform a remote surgical operation in an isolated room 91, in which a patient in, for example, an aseptic condition and a patient having been exposed to radiation are isolated. Arranged on a table 93 adjacent to a bed 92 in the isolated room 91 are a hook type treatment jig 21 and a scissors type treatment jig 35, which constitute a plurality of surgical instruments for a surgical operation through remote manipulation. The interconnection unit 24 constructed in the above-mentioned manner and disposed at an arm tip end 95 extended from a manipulator body 94 adapted for remote manipulation makes a trocar type treatment jig 37, which constitutes a surgical instrument, detachable.

Thereby, it is possible to arbitrarily and rapidly exchange a desired surgical instrument without contacting a patient in an aseptic condition and a patient who is impossible to contact, and to perform a surgical operation efficiently.

As described above, according to the present invention, a plurality of surgical instruments are readily exchanged during operations and a contact-free insulating configuration is provided. Therefore, a surgical operation apparatus and a surgical operation system, which are inexpensive and capable of securing sterilization, can be realized.

Although the invention has been described with respect to a specific embodiment for a complete and clear disclosure, the appended claims are not to be thus limited but are to be construed as embodying all modifications and alternative constructions that may occur to one skilled in the art which fairly fall within the basic teaching herein set forth.

What is claimed is:

1. A surgical operation apparatus comprising:
a surgical instrument;
a drive device that generates an energy with which the surgical instrument works;
an energy transmission cable that has a first end and a second end, and that transmits the energy, the first end being connected to the drive device;
a first connector provided on the second end;
an energy release unit that releases the energy transmitted by the electric transmission cable out of the first connector;
a second connector that is detachable from the first connector;
an energy receiving unit that receives the energy released from the energy release unit, and
an operation functioning unit that functions based on the energy received in the energy receiving unit.

2. The surgical operation apparatus according to claim 1, wherein
the energy release unit is disposed inside the first connector,
the second connector is provided in the surgical instrument,
the energy receiving unit is disposed inside the second connector, and
the operation functioning unit is provided in the surgical instrument.

3. The surgical operation apparatus according to claim 1, wherein the surgical instrument includes a probe that transmits the energy from the energy release unit to a biomedical tissue of a body to be operated.

4. The surgical operation apparatus according to claim 1, further comprising a latch that fixes the first connector on the second connector.

5. The surgical operation apparatus according to claim 1, further comprising:
a first magnetism generation unit that is provided in the first connector and that generates a first magnetism; and
a second magnetism generation unit that is provided in the second connector and that generates a second magnetism that attracts the first magnetism.

6. The surgical operation apparatus according to claim 1, further comprising:
an identification information storage unit that is disposed inside the second connector and that stores at least identification information of the surgical instrument;
a first information exchange unit that is disposed inside the first connector and that transmits a signal out of the first connector, the signal being used for storing information in the identification information storage unit and for reading the information from the identification information storage unit; and
a second information exchange unit that is disposed inside the second connector, that receives the signal to store the information in the identification information storage unit, and that reads the information from the identification information storage unit.

7. The surgical operation apparatus according to claim 6, wherein the drive device includes a control unit that controls generation of the energy with a drive parameter conformed to characteristics of the surgical instrument based on the identification information read by the first information exchange unit.

8. The surgical operation apparatus according to claim 1, wherein the surgical instrument includes
an ultrasonic vibrator that generates an ultrasonic vibration according to the energy received by the energy receiving unit; and
an ultrasonic vibration treatment unit that is vibrated by the ultrasonic vibration.

9. The surgical operation apparatus according to claim 1, wherein the surgical instrument includes
a treatment current generation unit that generates a high-frequency current for treatment according to the energy received by the energy receiving unit; and
a treatment electrode through which the current flows, the treatment electrode performing a high-frequency treatment on a body to be operated based on the current.

10. The surgical operation apparatus according to claim 1, wherein the drive device generates a first energy that is an electric energy as the energy,
the energy release unit is disposed inside the first connector, converts the first energy transmitted by the electric transmission cable into a second energy, and releases the second energy out of the first connector,
the energy receiving unit is disposed inside the second connector, receives the second energy, and converts the second energy into an electric energy, and
the operation functioning unit is provided on the surgical instrument and functions based on the electric energy converted by the energy receiving unit.

11. The surgical operation apparatus according to claim 1, wherein the surgical instrument is one of a laser scalpel, a microwave scalpel, a thermal scalpel, and an electric drill.

12. The surgical operation apparatus according to claim 1, further comprising:
a remote operation unit that performs a remote operation; and
a surgical operation manipulator that includes an arm that moves the surgical instrument to operate a body with the surgical instrument according to a command from the remote operation unit, wherein
the energy transmission cable is disposed on the surgical operation manipulator, and the first connector is disposed inside the arm.

13. The surgical operation apparatus according to claim 12, wherein the surgical instrument includes a probe that transmits the energy from the energy release unit to a biomedical tissue of a body to be operated.

14. The surgical operation apparatus according to claim 12, further comprising:
a first magnetism generation unit that is provided in the first connector and that generates a first magnetism;
a second magnetism generation unit that is provided in the second connector and that generates a second magnetism that attracts the first magnetism; and
a magnetism generation control unit that controls the first magnetism generated in the first magnetism generation unit and the second magnetism generated in the second magnetism generation unit.

15. The surgical operation apparatus according to claim 12, further comprising:
an identification information storage unit that is disposed inside the second connector and that stores at least identification information of the surgical instrument;
a first information exchange unit that is disposed inside the first connector and that transmits a signal out of the first connector, the signal being used for storing information in the identification information storage unit and for reading the information from the identification information storage unit; and
a second information exchange unit that is disposed inside the second connector, that receives the signal to store the information in the identification information storage unit, and that reads the information from the identification information storage unit.

16. The surgical operation apparatus according to claim 15, wherein the drive device includes a control unit that controls generation of the energy with a drive parameter conformed to characteristics of the surgical instrument based on the identification information read by the first information exchange unit.

17. The surgical operation apparatus according to claim 12, wherein the surgical instrument includes
an ultrasonic vibrator that generates an ultrasonic vibration according to the energy received by the energy receiving unit; and
an ultrasonic vibration treatment unit that is vibrated by the ultrasonic vibration.

18. The surgical operation apparatus according to claim 12, wherein the surgical instrument includes
a treatment current generation unit that generates a high-frequency current for treatment according to the energy received by the energy receiving unit; and
a treatment electrode through which the current flows, the treatment electrode performing a high-frequency treatment on a body to be operated based on the current.

19. The surgical operation apparatus according to claim 12, wherein the drive device generates a first energy that is an electric energy as the energy,
the energy release unit is disposed inside the first connector, converts the first energy transmitted by the electric transmission cable into a second energy, and releases the second energy out of the first connector,
the energy receiving unit is disposed inside the second connector, receives the second energy, and converts the second energy into an electric energy, and
the operation functioning unit is provided on the surgical instrument and functions based on the electric energy converted by the energy receiving unit.

20. The surgical operation apparatus according to claim 12, wherein the surgical instrument is one of a laser scalpel, a microwave scalpel, a thermal scalpel, and an electric drill.

21. A method of controlling a surgical operation apparatus that includes a first connector that releases energy generated by a drive device, a second connector that receives the energy, and a surgical instrument provided on the second connector, the method comprising:
reading information of the surgical instrument from an identification information storage unit provided in the second connector by using a first information exchange unit provided in the first connector; and
setting a drive state of the drive device based on the information.

22. The method according to claim 21, further comprising detecting interconnection between the first connector and the second connector.

23. The method according to claim 21, further comprising:
stopping reading of information by the first information exchange unit after the drive state of the drive device is set; and
generating the energy in the drive device based on the drive state after reading of information by the first information exchange unit is stopped.

24. The method according to claim 23, further comprising:
detecting drive information of the drive device;
transmitting the drive information through the first information exchange unit;
receiving the drive information transmitted, by a second information exchange unit provided in the second connector; and
storing the drive information received in the identification information storage unit.

25. The method according to claim 21, wherein the surgical instrument is selected from a plurality of surgical instruments.

26. The method according to claim 21, wherein the surgical instrument is one of a laser scalpel, a microwave scalpel, a thermal scalpel, and an electric drill.
